(19) United States
(12) Patent Application Publication
(10) Pub. No.: US 2002/0061525 A1
(43) Pub. Date: May 23, 2002

(54) Sequences of trail variants

(76) Inventors: Rodrigo Yelin, Tel Aviv (IL); Rami Khosravi, Herzliya (IL); Kinneret Savitzky, Tel Aviv (IL)

Correspondence Address: BIRCH STEWART KOLASCH & BIRCH, PO BOX 747, FALLS CHURCH, VA, 22040-0747, US

(21) Appl. No.: 09/855,544

(22) Filed: May 16, 2001

(30) Foreign Application Priority Data
May 16, 2000 (IL) 136156

(51) Int. Cl. A61K 48/00; C12Q 1/68; G01N 33/574; C07H 21/04; C12P 21/02; C12N 5/06

(52) U.S. Cl. 435/6; 514/44; 435/7.23; 435/69.1; 435/325; 435/320.1; 530/351; 536/23.5

(57) ABSTRACT

The present invention concerns novel, naturally occurring splice variants of the TNF-related apoptosis inducing ligand (TRAIL). Provided are coding nucleic acid sequences and the polypeptides encoded thereby, vectors and host cells containing said nucleic acid sequences and antibodies reactive with said polypeptides. The invention also concerns pharmaceutical compositions for the treatment of certain diseases, comprising the nucleic acid sequences, the amino acid sequences, the expression vectors or the antibodies.

The present application claims priority on Israeli Patent Application No. 136156 filed on May 16, 2000, which is herein incorporated by reference.

### FIELD OF THE INVENTION

The present invention concerns novel nucleic acid sequences, vectors and host cells containing them, amino acid sequences encoded by said sequences, and antibodies reactive with said amino acid sequences, as well as pharmaceutical compositions comprising any of the above. The present invention further concerns methods for screening for candidate agonists or antagonists utilizing said amino acid sequences.

### BACKGROUND OF THE INVENTION

Apoptosis, or programmed cell death, occurs during normal cellular differentiation and development of multicellular organisms. Apoptosis is induced by certain cytokines including TNF and Fas ligand in the TNF family through their death domain containing receptors, TNFRI and Fas. Another member in the TNF family has been identified and designated TRAIL (for TNF-related apoptosis inducing ligand) or Apo-2L (for Apo-2 ligand). Receptors for TRAIL include two death domain containing receptors, DR4 and DR5, as well as two decoy receptors, DcR1 and DcR2, lacking the intracellular signaling death domain.

TRAIL is a type II membrane protein and expressed in a variety of human tissues. Like TNF and Fas ligand, TRAIL induces apoptosis and NF-B activation in many tissues and cells.

The TRAIL may be expressed as a full length cell surface associated protein as well as in a soluble form. Both surface and soluble forms of TRAIL rapidly induce apoptosis on a wide range of cell lines.

Human TRAIL was first described in 1995 and is a 281aa. TRAIL's gene is located on chromosome 3 at location 3q26. TRAIL and its highly homologous cousin FasL/CD95L, regulate the immune system and are potent effector molecules for NK cell and T-cell cytotoxicity. T-cells that express TRAIL can mediate cytotoxicity through a perforin-independent, Fas-independent manner. Whereas Fas is upregulated by anti-CD3 mAb, TRAIL is not. However, IFN-significantly upregulates TRAIL expression on monocytes making them tumoricidal. This enhancement of TRAIL expression was a unique feature of type I IFNs (IFN-alpha and IFN-beta), and neither type II IFN (IFN-g) nor various other cytokines enhanced TRAIL expression on anti-CD3-stimulated cells.

TRAIL and Fas have variable expression in immune effectors. For instance, phenotypically immature NK cells CD161⁺/CD56⁻ mediate TRAIL-dependent but not FasL− or granule release-dependent cytotoxicity, whereas mature CD56⁺ NK cells mediate the latter two. Activated T-cells express both FasL and TRAIL. T-cells are highly sensitive towards FasL-induced apoptosis after prolonged activation in vitro, but remain completely resistant to TRAIL-induced apoptosis. Unlike FasL, whose transcripts are predminantly restricted to stimulated T-cells and sites of immune privilege, TRAIL mRNA is found widely expressed in a variety of tissue types, most notably spleen, lung, prostate, and lymphoid cells. Generally, TRAIL induces apoptosis in tumor cells whereas normal cells are relatively resistant without showing significant toxic side effects. Thus, TRAIL and the mechanisms for escape from TRAIL-induced apoptosis have been actively pursued in the oncology literature.

The tumoricidal effect of TRAIL was shown to be due to the rapid induction of tumor cell apoptosis. This effect is not limited to human mammary adenocarcinoma as TRAIL similarly suppressed the growth of TRAIL-sensitive colon carcinomas. These findings demonstrate that TRAIL rapidly exerts potent antitumor activity on TRAIL-sensitive tumors in vivo by selectively and directly activating tumor cell death (Walczak, et al. Tumoricidal activity of TRAIL in vivo, N/ature Med., 5:157-163 (1999)).

Two killer TRAIL receptors, DR4, DR5 were found, and other two protective receptors (decoy receptors) DcR1/TRID and DcR2 have been identified. Interestingly, cancer cell lines resistant to TRAIL show low expression levels of the killer receptors. Yet, the expression levels of those receptors increase upon exposure to chemotherapeutic agents such as etoposide, cis-platinum (CDDP) and doxorubicin, making the cells susceptible to TRAIL. Therefore a treatment combining antinepolatic drugs and the death ligand TRAIL is indeed promising.

Surface human TRAIL migrates at ˜32 kD by SDS-PAGE, soluble, human recombinant TRAIL migrates at 28 kD by SDS-PAGE and has also been observed in solution as a multimeric, (probably trimeric form) 80 kD form.

Glossary

In the following description and claims use will be made, at times, with a variety of terms, and the meaning of such terms as they should be construed in accordance with the invention is as follows:

“TRAIL VARIANT nucleic acid sequence”—the sequence shown in any one of SEQ ID NO: 1 to SEQ ID NO: 8, sequences having at least 90% identity (see below) to said sequence andfragments (see below) of the above sequences of least 20 b.p. long. These sequences are sequences coding for a novel, naturally occurring, alternative splice variants of the native and known TRAIL, depicted in the GenBank as AAC50332 under Accession Number G1:1149558. It should be emphasized that the novel variants of the present invention are naturally occurring sequences resulting from alternative splicing of the TRAIL gene and not merely truncated, mutated or fragmented forms of the gene.

The sequences of SEQ ID NOS: 1, 2, 3, 4 and 7 code for the TRAIL protein lack the conserved TNF domain. The sequences of SEQ ID NOS. 5, 6 and 8 lack part but not all of the TNF conserved domain and maintain its reading frame.

“TRAIL Variant product” also referred at times as the “TRAIL variant protein” or “TRAIL variant polypeptide”—is an amino acid sequence encoded by the RAIL variant nucleic acid sequences which is a naturally occurring mRNA sequence obtained as a result of alternative splicing. The amino acid sequence may be a peptide, a protein, as well as peptides or proteins having chemically modified amino acids (see below) such as a glycopeptide or glycoprotein. An example of the TRAIL variant products are shown in any one of SEQ ID NO: 9 or SEQ ID NO: 16. The term also includes homologues (see below) of said sequences in which one or more amino acids has been added, deleted, substituted (see below) or chemically modified (see below) as well as fragments (see below) of this sequence having at least 10 amino acids.

“Nucleic acid sequence”—a sequence composed of DNA nucleotides, RNA nucleotides or a combination of both types and may includes natural nucleotides, chemically modified nucleotides and synthetic nucleotides.

“Amino acid sequence”—a sequence composed of any one of the 20 naturally appearing amino acids, amino acids which have been chemically modified (see below), or composed of synthetic amino acids.

“Fragment of TRAIL variant nucleic acid sequence”—novel short stretch of nucleic acid sequences of at least 20 b.p., which does not appear as a continuous stretch in the original TRAIL sequence (see below). The fragment may be a sequence which was previously undescribed in the context of the published TRAIL RNA and which affects the amino acid sequence encoded by the known gene. For example, where the variant nucleic includes a sequence which was not included in the original TRAIL sequence (a sequence but which was an intron in the original sequence) the fragment is that additional sequence. The fragment may also be a region which is not an intron, which was not present in the original TRAIL sequence. Another example is when the TRAIL variant lacks a non-terminal region which was present in the original sequence. The two stretches of nucleotides spanning this region (upstream and downstream) are brought together by splicing in the TRAIL variant, but are spaced from each by the region in the original sequence and are thus not continuous.

“Fragments of TRAIL variant products”—novel amino acid sequences coded by the “fragment of TRAIL variant nucleic acid sequence” defined above.

“Homologues of variants”—amino acid sequences of variants in which one or more amino acids has been added, deleted or replaced. The addition, deletion or replacement should be in regions or adjacent to regions where the TRAIL variant differs from the original TRAIL sequence (see below).

“Conservative substitution”—refers to the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. [Six general classes of amino acid side chains have been categorized and include: Class I (Cys); Class II (Ser, Thr, Pro, Ala, Gly); Class III (Asn, Asp, Gin, Glu); Class IV (His, Arg, Lys); Class V (Ile, Leu, Val, Met); and Class VI (Phe, Tyr, Trp). For example, substitution of an Asp for another class III residue such as Asn, Gin, or Glu, is a conservative substitution.

“Non-conservative substitution”—refers to the substitution of an amino acid in one class with an amino acid from another class; for example, substitution of an Ala, a class II residue, with a class III residue such as Asp, Asn, Glu, or Gin.

“Chemically modified”—when referring to the product of the invention, means a product (protein) where at least one of its amino acid resides is modified either by natural processes, such as processing or other post-translational modifications, or by chemical modification techniques which are well known in the art. Among 5 the numerous known modifications typical, but not exclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

“Biologically active”—refers to the variant product having some sort of biological activity, for example, some physiologically measurable effect on target cells, molecules or tissues. In a specific example the biological activity refers to induction of apoptosis in a target cell population and in particular in cancer cells.

“Immunologically active” defines the capability of a natural, recombinant or synthetic varient product, or any fragment thereof, to induce a specific immune response in appropriate animals or cells and to bind with specific antibodies. Thus, for example, an immunologically active fragment of variant product denotes a fragment which retains some or all of the immunological properties of the variant product, e.g can bind specific anti-variant product antibodies or which can elicit an immune response which will generate such antibodies or cause proliferation of specific immune cells which produce variant.

“Optimal alignment”—is defined as an alignment giving the highest percent identity score. Such alignment can be performed using a variety of commercially available sequence analysis programs, such as the local alignment program LALIGN using a ktup of 1, default parameters and the default PAM. A preferred alignment is the one performed using the CLUSTAL-W program from 30 MacVector (TM), operated with an open gap penalty of 10.0, an extended gap penalty of 0.1, and a BLOSUM similarity matrix. If a gap needs to be inserted into a first sequence to optimally align it with a second sequence, the percent identity is calculated using only the residues that are paired with a corresponding amino acid residue (i.e., the calculation does not consider residues in the second sequences that are in the “gap” of the first sequence). In case of alignments of known gene sequences with that of the new variant, the optimal alignment invariably included aligning the identical parts of both sequences together, then keeping apart and unaligned the sections of the sequences that differ one from the other.

“Having at least 90% identity”—with respect to two amino acid or nucleic acid sequence sequences, refers to the percentage of residues that are identical in the two sequences when the sequences are optimally aligned. Thus, 90% amino acid sequence identity means that 90% of the amino acids in two or more optimally aligned polypeptide sequences are identical, however this definition explicitly excludes sequences which are 100% identical with the original sequence from which the variant of the invention was varied.

“Isolated nucleic acid molecule having an variant nucleic acid sequence”—is a nucleic acid molecule that includes the coding variant nucleic acid sequence. Said isolated nucleic acid molecule may include the variant nucleic acid sequence as an independent insert; may include the variant nucleic acid sequence fused to an additional coding sequences, encoding together a fusion protein in which the variant coding sequence is the dominant coding sequence (for example, the additional coding sequence may code for a signal peptide); the variant nucleic acid sequence may be in combination with non-coding sequences, e.g., introns or control elements, such as promoter and terminator elements or 5′ and/or 3′ untranslated regions, effective for expression of the coding sequence in a suitable host; or may be a vector in which the variant protein coding sequence is a heterologous.

“Expression vector”—refers to vectors that have the ability to incorporate and express heterologous DNA fragments in a foreign cell. Many prokaryotic and eukaryotic expression vectors are known and/or commercially available. Selection of appropriate expression vectors is within the knowledge of those having skill in the art.

“Deletion”—is a change in either nucleotide or amino acid sequence in which one or more nucleotides or amino acid residues, respectively, are absent.

“Insertion” or “addition”—is that change in a nucleotide or amino acid sequence which has resulted in the addition of one or more nucleotides or amino acid residues, respectively, as compared to the naturally occurring sequence.

“Substitution”—replacement of one or more nucleotides or amino acids by different nucleotides or amino acids, respectively. As regards amino acid sequences the substitution may be conservative or non-conservative.

“Antibody”—refers to IgG, IgM, IgD, IgA, and IgG antibody. The definition includes polyclonal antibodies or monoclonal antibodies. This term refers to whole antibodies or fragments of the antibodies comprising the antigen-binding domain of the anti-variant product antibodies, e.g. antibodies without the Fe portion, single chain antibodies, fragments consisting of essentially only the variable, antigen-binding domain of the antibody, etc.

“Agonist”—as used herein, refers to a molecule which activates cells, in a similar manner to the natural TRAIL molecule and the TRAIL variants of the invention. For example, a molecule which may induce apoptosis is a manner similar to that of the TRAIL. Agonists may be polypeptides, nucleic acids, carbohydrates, lipids, or derivatives thereof, or any other molecules which can bind to and activate the variant product.

“Antagonists”—refers to a molecule which inhibits the activity of the TRAIL variant of the invention. This may be done by any mechanism known to antagonists or inhibit biological receptor such as block of the receptor, block of active site of the ligand, competition on binding site in ligand, enhancement of degradation, etc. Antagonist may be polypeptides, nucleic acids, carbohydrates, lipids, or derivatives thereof, or any other molecules which bind to and modulate the activity of said product.

“Treating a disease”—refers to administering a therapeutic substance effective to ameliorate symptoms associated with a disease, to lessen the severity or cure the disease, or to prevent the disease from occurring. In a specific example the disease is cancer or a degenerative disease.

“Target cell population”—a population of a specific type of cells which should be destroyed by the cytotoxic effect of the TRAIL variant of the invention. A specific example is cancer cells.

“Detection”—refers to a method of detection of a disease, disorder, pathological or normal condition. This term may refer to detection of a predisposition to a disease as well as for establishing the prognosis of the patient by determining the severity of the disease.

“Probe”—the variant nucleic acid sequence, or a sequence complementary therewith, when used to detect presence of other similar sequences in a sample. The detection is carried out by identification of hybridization complexes between the probe and the assayed sequence. The probe may be attached to a solid support or to a detectable label.

“Original TRAIL sequence”—the amino acid or nucleic acid sequence from which the TRAIL variants of the invention have been varied as a result of alternative slicing. The original sequence is the sequence of the Apo2L depicted as AAC50332 in the GenBank under Accession Number GI:1149558.

### SUMMARY OF THE INVENTION

The present invention is based on the finding of eight novel, naturally occurring splice variants of the TNF-related apoptosis inducing ligand (TRAIL), which are naturally occurring sequences obtained by alternative splicing of the known TRAIL genes depicted depicted as AAC50332 in the GenBank under Accession Number GI:1149558. The novel splice variants of the invention are not merely truncated forms, fragments or mutations of known gene, but rather novel sequences which naturally occur within the body of individuals. Some of the variants of the invention completely lack the conserved TNF domain and others lack part of it.

The term “alternative splicing” in the context of the present invention and claims refers to: intron inclusion, exon exclusion, addition or deletion of terminal sequences in the variant as compared to the original sequences.

The novel TRAIL variant products of the invention may have the same physiological activity as the original TRAIL from which they are varied (although perhaps at a different level); may have an opposite physiological activity from the activity featured by the original peptide from which they are varied; may have a completely different, unrelated activity to the activity of the original from which they are varied; or alternatively may have no activity at all and this may lead to various diseases or pathological conditions.

In addition, in all the above cases, (i.e. where the variant has the same or different activity), the variant may differ from the original peptide in its cellular distribution, the timing of its expression, its rate of clearance and degradation, the ligand binding activities, the regulation of expression, as well as other physiological properties not directly related to the activities on target cells or tissues.

The novel TRAIL variants may also serve for detection purposes, i.e. their presence or level may be indicative of a disease, disorder, pathological or normal condition involving typically non-normal apoptosis (excess apoptosis or lack of) such as cancer, degenerative conditions; diseases involving the immune system and diseases involved in non-normal development of tissues. Alternatively the ratio between the level variants and the level original TRAIL peptide from which they were varied, or the ratio of the variants to each other may be indicative to such a disease, disorder, pathological or normal condition.

For example, for detectional purposes, it is possible to establish differential expression of the variants in various tissues as compared to each other and as compared to the original TRAIL sequence. One variant may be expressed mainly in one tissue, while the original TRAIL sequence from which it has been varied, or the other variants may, be expressed mainly in another tissue. Understanding of the distribution of the two variants in various tissues may be helpful in basic research, for understanding the physiological function of the gene as well as may help in targeting pharmaceuticals or developing pharmaceuticals.

The study of the variants may also be helpful to distinguish various stages in the life cycles of the same type of cells which may also be helpful for development of pharmaceuticals for various pathological conditions in which cell cycles is abnonnal, notably cancer, but also in various conditions involving degeneration of cells such as aging and developmental conditions (wherein apoptosis is a part of the normal development).

Thus the detection may by determination of the presence or the level of expression of the variant within a specific cell population, comprising said presence or level between various cell types in a tissue, between different tissues and between individuals.

Thus the present invention provides by its first aspect, a novel isolated nucleic acid molecule comprising or consisting of any one of the coding sequence SEQ ID NO: 1 to SEQ ID NO: 8, fragments of said coding sequence having at least 20 nucleic acids (provided that said fragments are continuous stretches of nucleotides not present in the original TRAIL sequence from which the variant was varied), or a molecule comprising a sequence having at least 90%, identity to SEQ ID NO:1 to SEQ ID NO: 8.

The present invention further provides a protein or polypeptide comprising or consisting of an amino acid sequence encoded by any of the above nucleic acid sequences, termed herein “TRAIL variant product”, for example, an amino acid sequence having the sequence as depicted in any one of SEQ ID NO:9 to SEQ ID NO: 16, fragments of the above amino acid sequence having a length of at least 10 amino acids coded by the above fragments of the nucleic acid sequences, as well as homologues of the above amino acid sequences in which one or more of the amino acid residues has been substituted (by conservative or non-conservative substitution) added, deleted, or chemically modified.

The deletions, insertions and modifications should be in regions, or adjacent to regions, wherein the variant differs from the original sequence, and these regions are explained in detail in the “Glossary” part of the specification.

For example, where the variant is different from the original sequence by addition of a short stretch of additional amino, the invention also concerns homologues of that variant where this additional short stretch is altered, some of these alterations being conservative or non-conservative substitutions of the original additional amino acids of the novel variants. In all cases the changes in the homolog, as compared to the original sequence, are in the same regions where the variant differs from the original sequence, or in regions adjacent to said region.

Another example is where the variant lacks a non-terminal region which is present in the original sequence. The homologues may lack in the same region a smaller number of amino acids than the parent variant. Again the deletion is in the same region where the variant lacks a sequence as compared to the original sequence, or in a region adjacent thereto.

It should be appreciated that once a man versed in the art's attention is directed to the importance of a specific region, due to the fact that this region differs in the TRAIL variant as compared to the original TRAIL sequence, there is no problem in derivating said specific region by addition to it, deleting from it, or substituting some amino acids in it. Thus homologues of the TRAIL variants which are derivated from the original TRAIL by changes (deletion, addition, substitution) only in said region as well as in regions adjacent to it are also a part of the present invention. Generally, if the TRAIL variant is distinguished from the original TRAIL sequence by some sort of physiological activity, then the homolog is distinguished from the original TRAIL sequence in essentially the same manner.

The present invention further provides nucleic acid molecule comprising or consisting of a sequence which encodes the above amino acid sequences, (including the fragments and homologues of the amino acid sequences). Due to the degenerative nature of the genetic code, a plurality of alternative nucleic acid sequences, beyond those depicted in any one of SEQ ID NO:1 to SEQ ID NO:8, can code for the amino acid sequence of the invention. Those alternative nucleic acid sequences which code for the same amino acid sequences depicted in sequence SEQ ID NO:9 to SEQ ID NO: 16 are also an aspect of the of the present invention.

The present invention further provides expression vectors and cloning vectors comprising any of the above nucleic acid sequences, as well as host cells transfected by said vectors.

The present invention still further provides pharmaceutical compositions comprising, as an active ingredient, said nucleic acid molecules, said expression vectors, or said protein or polypeptide.

These pharmaceutical compositions are suitable for the treatment of diseases and pathological conditions, which can be ameliorated or cured by raising the level of any one of the variant products of the invention. In particular these diseases are such where a beneficial effect is evident by inducing apoptosis in a target cell population and in particular in cancer cells. As explained in the Background section of the specification, a unique feature of the TRAIL protein is its ability to cause a cytotoxic effect in cancer cells (by induction of apoptosis) while not substantially damaging normal cells. The pharmaceutical composition may also be used in connection with various immunological diseases and disorders, in rheumatology as well as in conjunction with transplantation.

By a second aspect, the present invention provides a nucleic acid molecule comprising or consisting of a non-coding sequence which is complementary to that of any one of SEQ ID NO:1 to SEQ ID NO:8, or complementary to a sequence having at least 90% identity to said sequence or a fragment of said two sequences (according to the above definition of fragment). The complementary sequence may be a DNA sequence which hybridizes with any one of SEQ of ID NO:1 to SEQ ID NO:8 or hybridizes to a portion of that sequence having a length sufficient to inhibit the transcription of the complementary sequence. The complementary sequence may be a DNA sequence which can be transcribed into an mRNA being an antisense to the mRNA transcribed from any one of SEQ ID NO: 1 to SEQ ID NO:8 or into an mRNA which is an antisense to a fragment of the mRNA transcribed from any one of SEQ ID NO: 1 to SEQ ID NO:8 which has a length sufficient to hybridize with the mRNA transcribed from SEQ ID NO: 1 to SEQ ID NO: 8, so as to inhibit its translation. The complementary sequence may also be the mRNA or the fragment of the mRNA itself.

The nucleic acids of the second aspect of the invention may be used for therapeutic or diagnostic applications for example as probes used for the detection of the TRAIL variants of the invention. The presence of the TRAIL variant transcript or the level of the variant transcript may be indicative of a multitude of diseases, disorders and various pathological as well as normal conditions and in particular indicative of cancer or a degenerative condition. In addition, the ratio of the level of the transcripts of the variants of the invention may also be compared to that of the transcripts of the original TRAIL sequences from which they were varied, or to the level of transcript of each other, and said ratio may be indicative to a multitude of diseases, disorders and various pathological and normal conditions.

The present invention also provides expression vectors comprising any one of the above defined complementary nucleic acid sequences and host cells transfected with said nucleic acid sequences or vectors, being complementary to those specified in the first aspect of the invention.

The invention also provides anti-variant product antibodies, namely antibodies directed against the TRAIL variant product which specifically bind to said TRAIL variant product. Said antibodies are useful both for diagnostic and therapeutic purposes. For example said antibodies may be as an active ingredient in a pharmaceutical composition as will be explained below.

The present invention also provides pharmaceutical compositions comprising, as an active ingredient, the nucleic acid molecules which comprise or consist of said complementary sequences, or of a vector comprising said complementary sequences. The pharmaceutical composition thus provides pharmaceutical compositions comprising, as an active ingredient, said anti-variant product antibodies.

The pharmaceutical compositions comprising said anti-variant product antibodies or the nucleic acid molecule comprising said complementary sequence, are suitable for the treatment of diseases and pathological conditions where a therapeutically beneficial effect may be achieved by neutralizing the variants (either at the transcript or product level) or decreasing the amount of the variant product or blocking its binding to its ligand, for example, by the neutralizing effect of the antibodies, or by the decrease of the effect of the antisense mRNA in decreasing expression level of the TRAIL variant product.

In particular, these diseases are manifested by undesired death, due to apoptosis processes, and may include various degenerative diseases leading to neurodegenerative processes, autoimmune diseases and various undesired effects of aging.

According to the third aspect of the invention the present invention provides methods for detecting the level of the transcript (mRNA) of said TRAIL variants product in a body fluid sample, or in a specific tissue sample, for example by use of probes comprising or consisting of said coding sequences; as well as methods for detecting levels of expression of said product in tissue, e.g. by the use of antibodies capable of specifically reacting with the variant products of the invention. Detection of the level of the expression of the variant of the invention in particular as compared to that of the original sequence from which it was varied or compared to other variant sequences all varied from the same original sequence may be indicative of a plurality of physiological or pathological conditions.

The method, according to this latter aspect, for detection of a nucleic acid sequence which encodes the TRAIL variant products in a biological sample, comprises the steps of:

(a) providing a probe comprising at least one of the nucleic acid sequences defined above;

(b) contacting the biological sample with said probe under conditions allowing hybridization of nucleic acid sequences thereby enabling formation of hybridization complexes;

(c) detecting hybridization complexes, wherein the presence of the complex indicates the presence of nucleic acid sequence encoding the TRAIL variant product in the biological sample.

The method as described above is qualitative, i.e. indicates whether the transcript is present in or absent from the sample. The method can also be quantitative, by determining the level of hybridization complexes and then calibrating said levels to determining levels of transcripts of the desired variant in the sample.

Both qualitative and quantitative determination methods can be used for diagnostic, prognostic and therapy planning purposes.

By a preferred embodiment the probe is part of a nucleic acid chip used for detection purposes, i.e. the probe is a part of an array of probes each present in a known location on a solid support.

The nucleic acid sequence used in the above method may be a DNA sequence an RNA sequence, etc; it may be a coding or a sequence or a sequence complementary thereto (for respective detection of RNA transcripts or coding-DNA sequences). By quantization of the level of hybridization complexes and calibrating the quantified results it is possible also to detect the level of the transcript in the sample.

Methods for detecting mutations in the region coding for the TRAIL variant product are also provided, which may be methods carried-out in a binary fashion, namely merely detecting whether there is any mismatches between the normal variant nucleic acid sequence of the invention and the one present in the sample, or carried-out by specifically detecting the nature and location of the mutation.

The present invention also concerns a method for detecting the TRAIL variant product in a biological sample, comprising the steps of:

(a) contacting with said biological sample the antibody of the invention, thereby forming an antibody-antigen complex; and

(b) detecting said antibody-antigen complex

wherein the presence of said antibody-antigen complex correlates with the presence of the TRAIL variant product in said biological sample.

As indicated above, the method can be quantitized to determine the level or the amount of the TRAIL variant in the sample, alone or in comparison to the level of the original TRAIL amino acid sequence from which it was varied, and qualitative and quantitative results may be used for diagnostic, prognostic and therapy planning purposes.

By yet another aspect the invention also provides a method for identifying candidate compounds capable of binding to the variant product and modulating its activity (being either agonists or antagonists). The method includes:

(i) providing a protein or polypeptide comprising an amino acid sequence substantially as depicted in any one of SEQ ID NO:1 or SEQ ID NO:2, or a fragment of such a sequence;

(ii) contacting a candidate compound with said amino acid sequence;

(iii) measuring the physiological effect of said candidate compound on the activity of the amino acid sequences and selecting those compounds which show a significant effect on said physiological activity.

The present invention also concerns compounds identified by the above methods described above, which compound may either be an agonist or antagonist of the TRAIL variant product.

### BRIEF DESCRIPTION OF THE DRAWINGS

In order to better understand the invention and to see how it may be carried out in practice, some embodiments will now be described, by way of non-limiting examples only, with reference to the accompanying drawings in which:

FIG. 1 is alignment of the amino acid of SEQ ID NO:9 to the original TRAIL protein;

FIG. 2 is alignment of the amino acid of SEQ ID NO:10 to the original TRAIL protein;

FIG. 3 is alignment of the amino acid of SEQ ID NO:11 to the original TRAIL protein;

FIG. 4 is alignment of the amino acid of SEQ ID NO:12 to the original TRAIL protein;

FIG. 5 is alignment of the amino acid of SEQ ID NO:13 to the original TRAIL protein;

FIG. 6 is alignment of the amino acid of SEQ ID NO:14 to the original TRAIL protein;

FIG. 7 is alignment of the amino acid of SEQ ID NO:15 to the original TRAIL protein;

FIG. 8 is alignment of the amino acid of SEQ ID NO:16 to the original TRAIL protein;

FIG. 9 shows multiple alignment of all eight variants of TRAIL (depicted in SEQ ID NO:9 to SEQ ID NO:16) to each other; and

FIG. 10 shows a schematic representation of wild type TRAIL (ups), of the truncated variants (SEQ ID NOS.9, 10, 11, 12 and 15) and of the deletion variants (SEQ ID NOS: 13, 14 and 16).

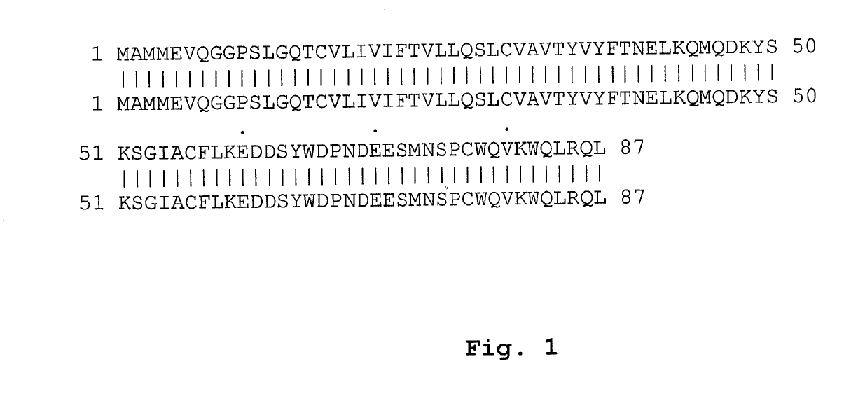

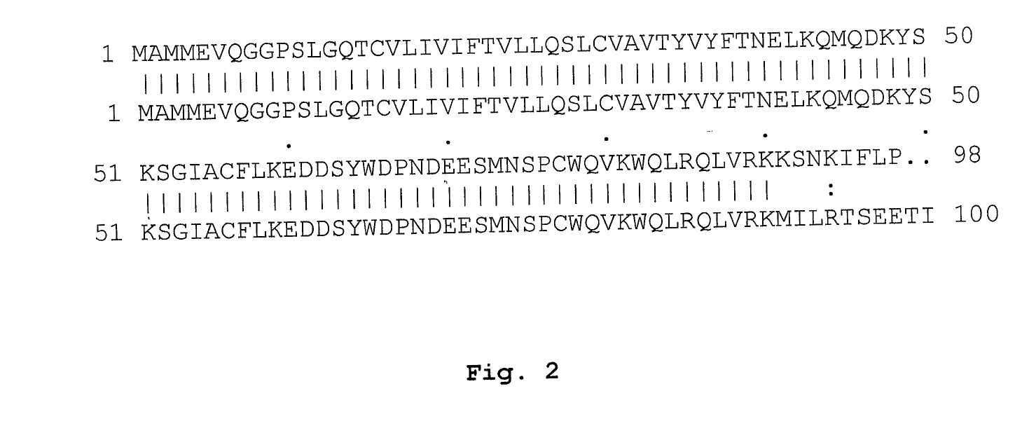

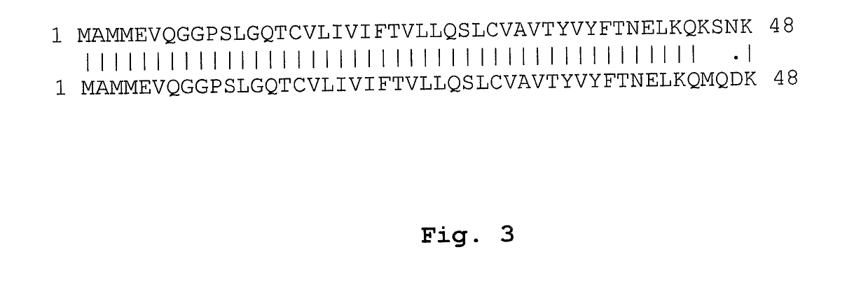

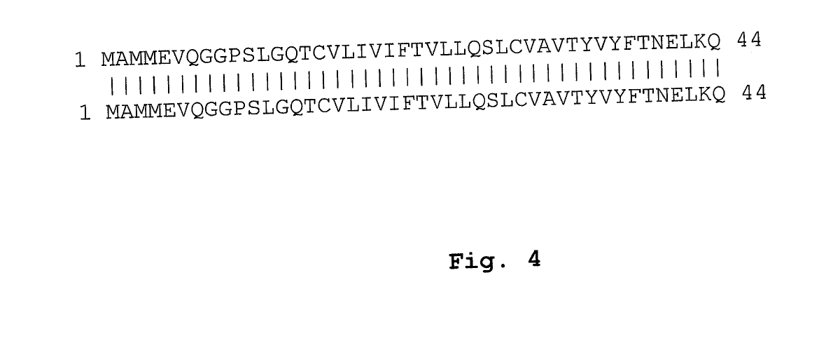

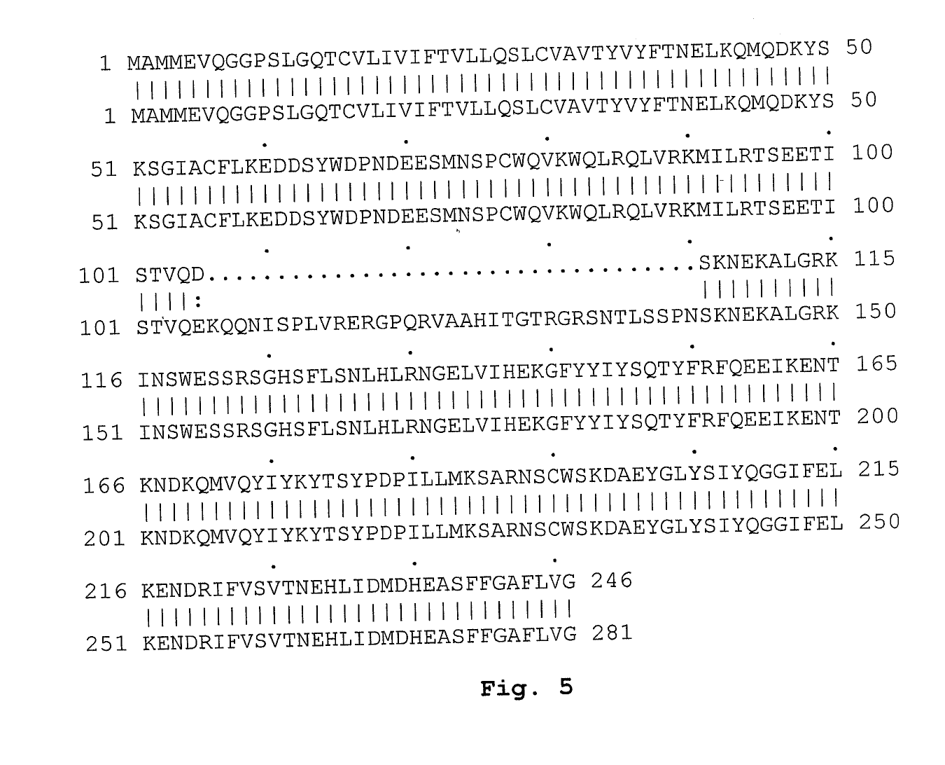

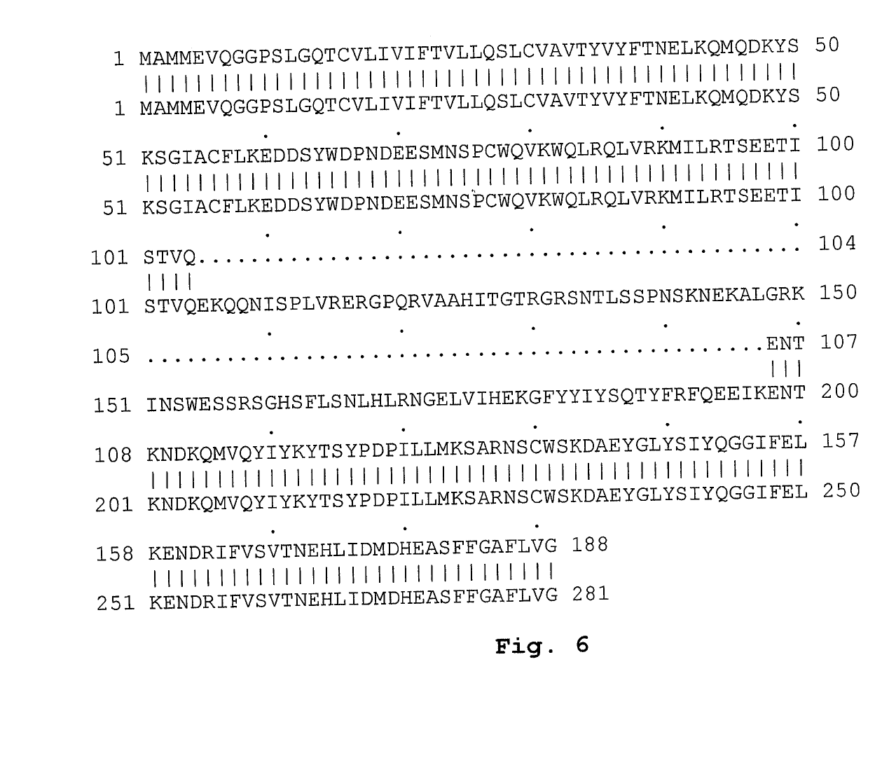

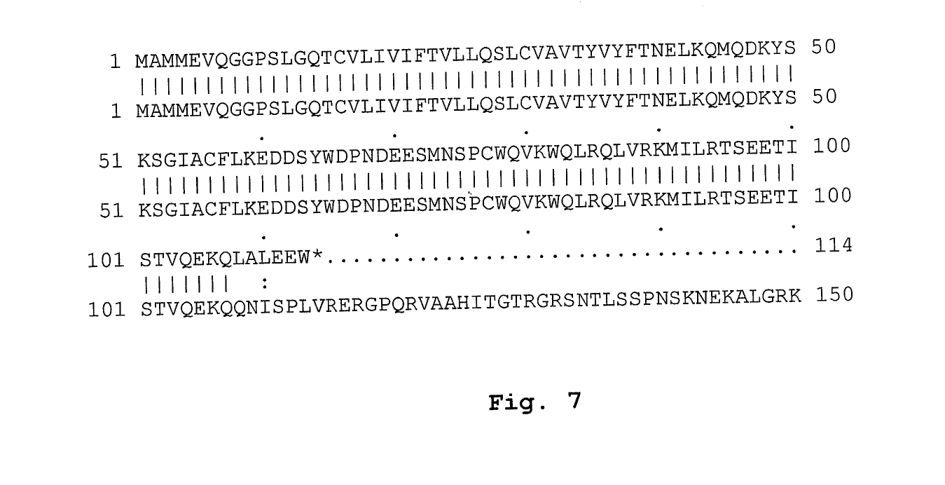

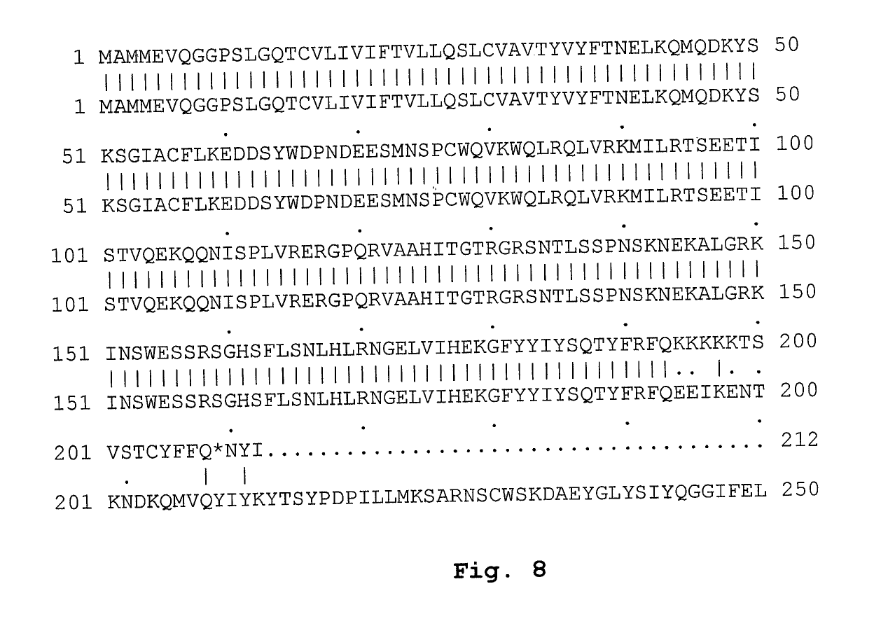

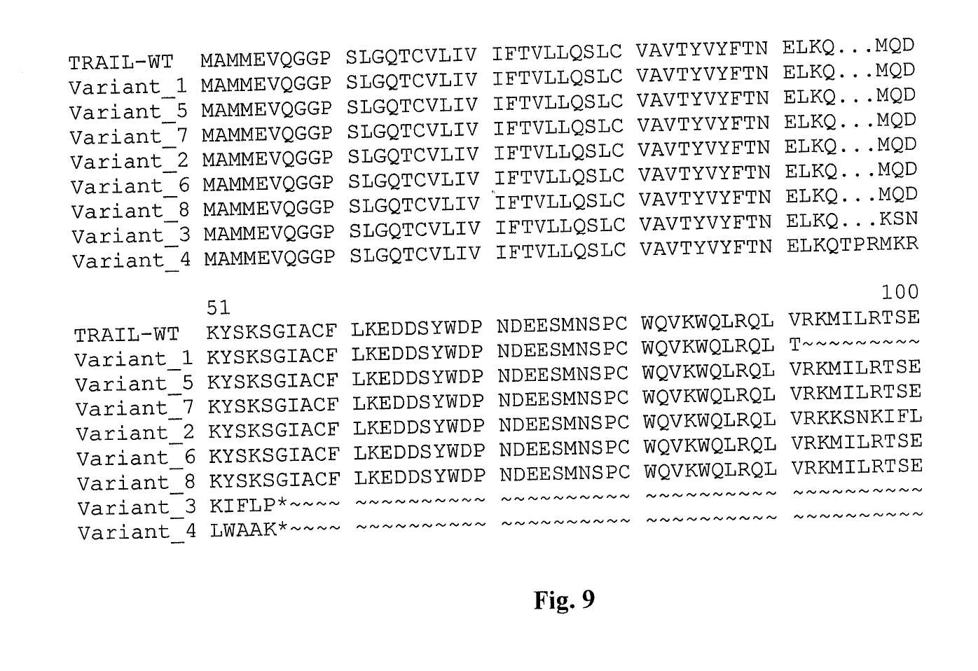

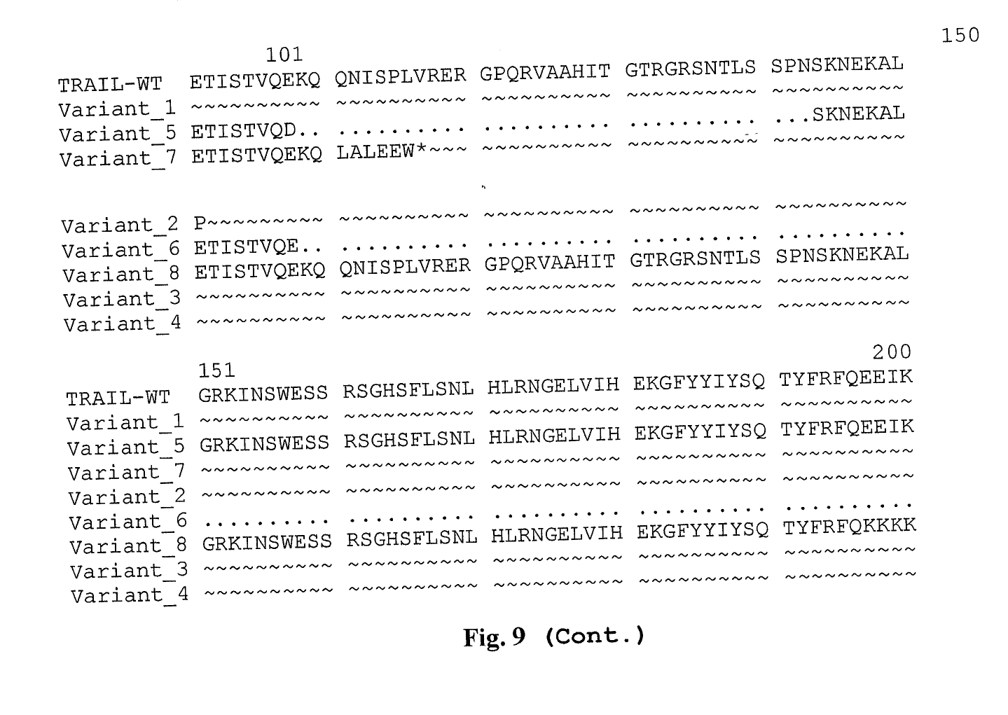

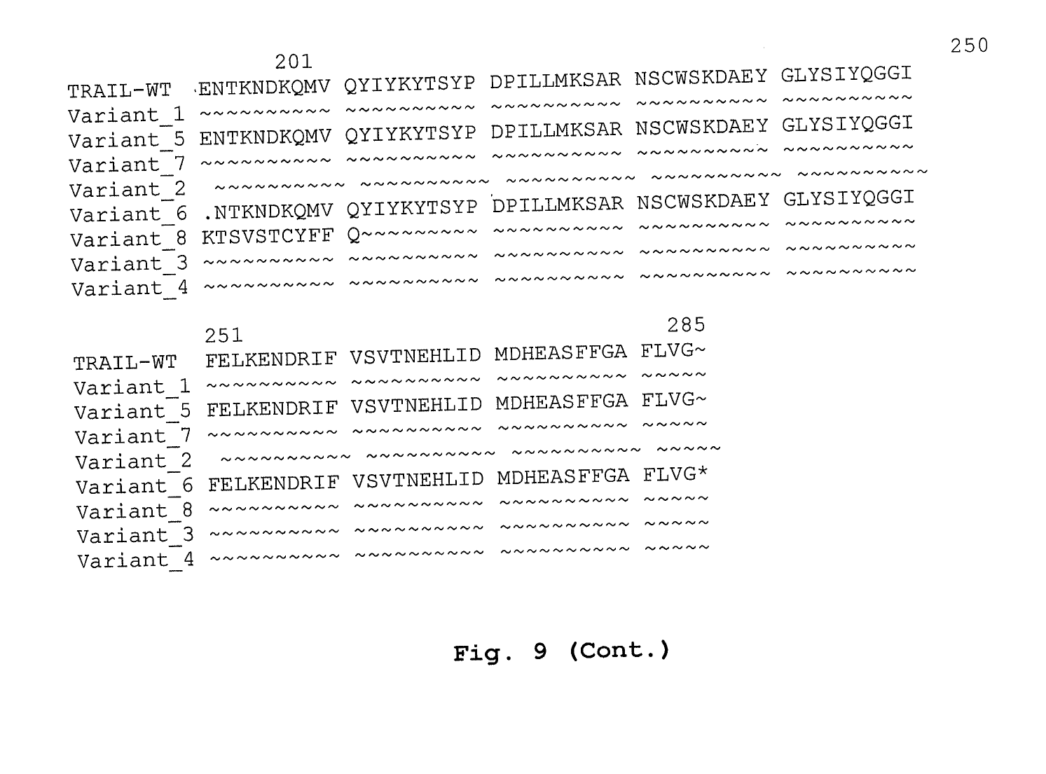

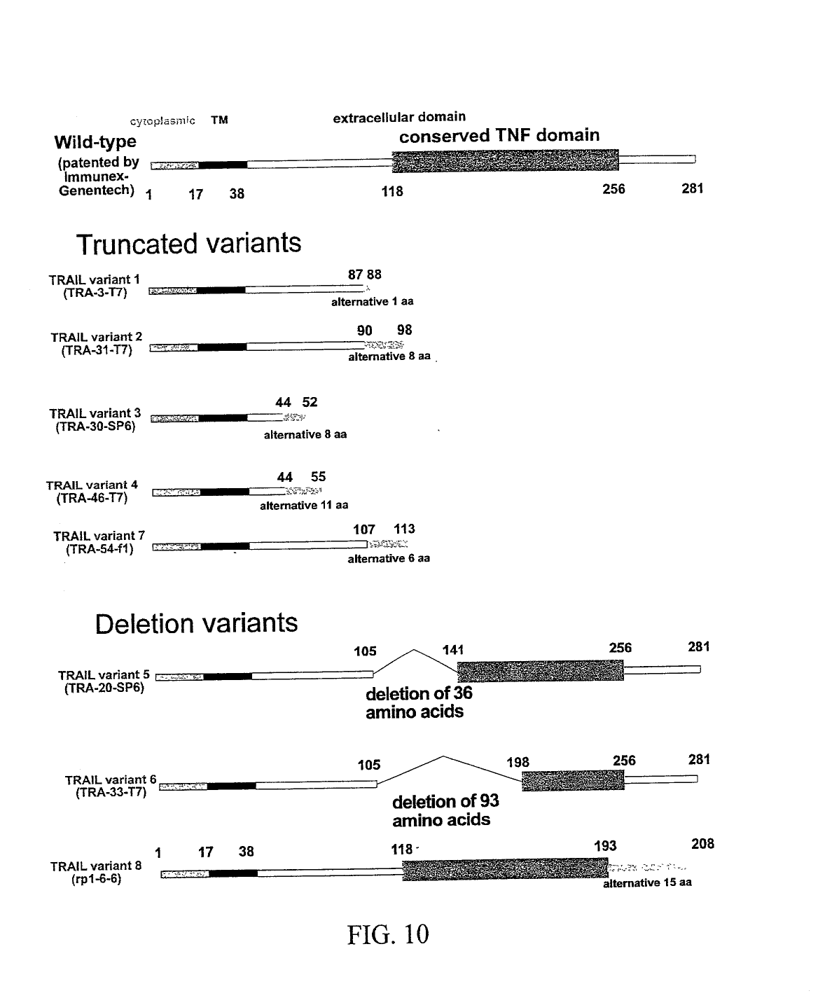

### DETAILED DESCRIPTION OF PREFERRED EMBODIMENTS

### EXAMPLE I

### TRAIL Variant Nucleic Acid Sequence

The nucleic acid sequences of the invention include nucleic acid sequences which encode TRAIL variant product and fragments and analogs thereof. The nucleic acid sequences may alternatively be sequences complementary to the above coding sequence, or to a region of said coding sequence. The length of the complementary sequence is sufficient to avoid the expression of the coding sequence. The nucleic acid sequences may be in the form of RNA or in the form of DNA, and include messenger RNA, synthetic RNA and DNA, cDNA, and genomic DNA. The DNA may be double-stranded or single-stranded, and if single-stranded may be the coding strand or the non-coding (anti-sense, complementary) strand. The nucleic acid sequences may also both include dNTPs, rNTPs as well as non naturally occurring sequences. The sequence may also be a part of a hybrid between an amino acid sequence and a nucleic acid sequence.

In a general embodiment, the nucleic acid sequence has at least 90%, identity with any one of the sequence identified as SEQ ID NO:1 to SEQ ID NO:8.

The nucleic acid sequences may include the coding sequence by itself. By another alternative the coding region may be in combination with additional coding sequences, such as those coding for fusion protein or signal peptides, in combination with non-coding sequences, such as introns and control elements, promoter and terminator elements or 5′ and/or 3′ untranslated regions, effective for expression of the coding sequence in a suitable host, and/or in a vector or host environment in which the variant nucleic acid sequence is introduced as a heterologous sequence.

The nucleic acid sequences of the present invention may also have the product coding sequence fused in-frame to a marker sequence which allows for purification of the variant product. The marker sequence may be, for example, a hexahistidine tag to provide for purification of the mature polypeptide fused to the marker in the case of a bacterial host, or, the marker sequence may be a hemagglutinin (HA) tag when a mammalian host, e.g. COS-7 cells, is used. The HA tag corresponds to an epitope derived from the influenza hemagglutinin protein (Wilson, I., et al. Cell 37:767 (1984)).

Also included in the scope of the invention are fragments as defined above also referred to herein as oligonucleotides, typically having at least 20 bases, preferably 20-30 bases corresponding to a region of the coding-sequence nucleic acid sequence. The fragments may be used as probes, primers, and when complementary also as antisense agents, and the like, according to known methods.

As indicated above, the nucleic acid sequence may be substantially a depicted in any one of SEQ ID NO:1 to SEQ ID NO:8 or fragments thereof or sequences having at least 90% identity to the above sequence as explained above. Alternatively, due to the degenerative nature of the genetic code, the sequence may be a sequence coding for any one of the amino acid sequence of SEQ ID NO:9 to SEQ ID NO: 16, or fragments or analogs of said amino acid sequence.

A. Preparation of Nucleic Acid Sequences

The nucleic acid sequences may be obtained by screening cDNA libraries using oligonucleotide probes which can hybridize to or PCR-amplify nucleic acid sequences which encode the TRAIL variant products disclosed above. cDNA libraries prepared from a variety of tissues are commercially available and procedures for screening and isolating cDNA clones are well-known to those of skill in the art. Such techniques are described in, for example, Sambrook et al. (1989) Molecular Cloning: A Laboratory Manual (2nd Edition), Cold Spring arbor Press, Plainview, N.Y. and Ausubel FM et al. (1989) Current Protocols in Molecular Biology, John Wiley & Sons, New York, N.Y.

The nucleic acid sequences may be extended to obtain upstream and downstream sequences such as promoters, regulatory elements, and 5′ and 3′ untranslated regions (UTRs). Extension of the available transcript sequence may be performed by numerous methods known to those of skill in the art, such as PCR or primer extension (Sambrook et al., supra), or by the RACE method using, for example, the Marathon RACE kit (Clontech, Cat. # K1802-1).

Alternatively, the technique of “restriction-site” PCR (Gobinda et al. PCR Methods Applic. 2:318-22, (1993)), which uses universal primers to retrieve flanking sequence adjacent a known locus, may be employed. First, genomic DNA is amplified in the presence of primer to a linker sequence and a primer specific to the known region. The amplified sequences are subjected to a second round of PCR with the same linker primer and another specific primer internal to the first one. Products of each round of PCR are transcribed with an appropriate RNA polymerase and sequenced using reverse transcriptase.

Inverse PCR can be used to amplify or extend sequences using divergent primers based on a known region (Triglia, T. et al., Nucleic Acids Res. 16:8186, (1988)). The primers may be designed using OLIGO(R) 4.06 Primer Analysis Software (1992; National Biosciences Inc, Plymouth, Minn.), or another appropriate program, to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Capture PCR (Lagerstrom, M. et al., PCR Methods Applic. 1:111-19, (1991)) is a method for PCR amplification of DNA fragments adjacent to a known sequence in human and yeast artificial chromosome DNA. Capture PCR also requires multiple restriction enzyme digestions and ligations to place an engineered double-stranded sequence into a flanking part of the DNA molecule before PCR.

Another method which may be used to retrieve flanking sequences is that of Parker, J. D., et al., Nucleic Acids Res., 19:3055-60, (1991)). Additionally, one can use PCR, nested primers and PromoterFinder™ libraries to “walk in” genomic DNA (PromoterFinder™; Clontech, Palo Alto, Calif.). This process avoids the need to screen libraries and is useful in finding intron/exon junctions. Preferred libraries for screening for full length cDNAs are ones that have been size-selected to include larger cDNAs. Also, random primed libraries are preferred in that they will contain more sequences which contain the 5′ and upstream regions of genes.

A randomly primed library may be particularly useful if an oligo d(T) library does not yield a full-length cDNA. Genomic libraries are useful for extension into the 5′ nontranslated regulatory region.

The nucleic acid sequences and oligonucleotides of the invention can also be prepared by solid-phase methods, according to known synthetic methods. Typically, fragments of up to about 100 bases are individually synthesized, then joined to form continuous sequences up to several hundred bases.

The TRAIL variants of the invention were obtained by using RT-PCR from two tissues (B-lymphocytes and liver) and the different bands that were detected were cloned to a plasmid. 15 out of 70 PCR-tested positive, colonies were separated and the novel variants of the invention identified.

B. Use of TRAIL Variants Nucleic Acid Sequence for the Production of TRAIL Variant Products

In accordance with the present invention, nucleic acid sequences specified above may be used as recombinant DNA molecules that direct the expression of TRAIL variant products.

As will be understood by those of skill in the art, it may be advantageous to produce TRAIL variant product-encoding nucleotide sequences possessing codons other than those which appear in any one of SEQ ID NO:1 to SEQ ID NO:8 which are those which naturally occur in the human genome. Codons preferred by a particular prokaryotic or eukaryotic host (Murray, E. et al. Nuc Acids Res., 17:477-508, (1989)) can be selected, for example, to increase the rate of variant product expression or to produce recombinant RNA transcripts having desirable properties, such as a longer half-life, than transcripts produced from naturally occurring sequence.

The nucleic acid sequences of the present invention can be engineered in order to alter a TRAIL variant product coding sequence for a variety of reasons, including but not limited to, alterations which modify the cloning, processing and/or expression of the product. For example, alterations may be introduced using techniques which are well known in the art, e.g., site-directed mutagenesis, to insert new restriction sites, to alter glycosylation patterns, to change codon preference, etc.

The present invention also includes recombinant constructs comprising one or more of the sequences as broadly described above. The constructs comprise a vector, such as a plasmid or viral vector, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. Appropriate cloning and expression vectors for use with prokaryotic and eukaryotic hosts are also described in Sambrook, et al., (supra).

The present invention also relates to host cells which are genetically engineered with vectors of the invention, and the production of the product of the invention by recombinant techniques. Host cells are genetically engineered (i.e., transduced, transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the expression of the variant nucleic acid sequence. The culture conditions, such as temperature, pH and the like, are those previously used with the host cell selected for expression, and will be apparent to those skilled in the art.

The nucleic acid sequences of the present invention may be included in any one of a variety of expression vectors for expressing a product. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host. The appropriate DNA sequence may be inserted into the vector by a variety of procedures. In general, the DNA sequence is inserted into an appropriate restriction endonuclease site(s) by procedures known in the art. Such procedures and related sub-cloning procedures are deemed to be within the scope of those skilled in the art.

The DNA sequence in the expression vector is operatively linked to an appropriate transcription control sequence (promoter) to direct mRNA synthesis. Examples of such promoters include: LTR or SV40 promoter, the E.coli lac or trp promoter, the phage lambda PL promoter, and other promoters known to control expression of genes in prokaryotic or eukaryotic cells or their viruses. The expression vector also contains a ribosome binding site for translation initiation, and a transcription terminator. The vector may also include appropriate sequences for amplifying expression. In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E.coli.

The vector containing the appropriate DNA sequence as described above, as well as an appropriate promoter or control sequence, may be employed to transform an appropriate host to permit the host to express the protein. Examples of appropriate expression hosts include: bacterial cells, such as E. coli, Streptomyces, Salmonella typhimurium; fungal cells, such as yeast; insect cells such as Drosophila and Spodoptera Sf9; animal cells such as CHO, COS, HEK 293 or Bowes melanoma; adenoviruses; plant cells, etc. The selection of an appropriate host is deemed to be within the scope of those skilled in the art from the teachings herein. The invention is not limited by the host cells employed.

In bacterial systems, a number of expression vectors may be selected depending upon the use intended for the TRAIL variant product. For example, when large quantities of TRAIL variant product are needed for the induction of antibodies, vectors which direct high level expression of fusion proteins that are readily purified may be desirable. Such vectors include, but are not limited to, multifunctional E.coli cloning and expression vectors such as Bluescript(R) (Stratagene), in which the TRAIL variant polypeptide coding sequence may be ligated into the vector in-frame with sequences for the amino-terminal Met and the subsequent 7 residues of beta-galactosidase so that a hybrid protein is produced; pIN vectors (Van Heeke & Schuster J Biol. Chem. 264:5503-5509, (1989)); pET vectors (Novagen, Madison Wis.); and the like.

In the yeast Saccharomyces cerevisiae a number of vectors containing constitutive or inducible promoters such as alpha factor, alcohol oxidase and PGH may be used. For reviews, see Ausubel et al. (supra) and Grant et al., (Methods in Enzymology 153:516-544, (1987)).

In cases where plant expression vectors are used, the expression of a sequence encoding variant product may be driven by any of a number of promoters. For example, viral promoters such as the 35S and 19S promoters of CaMV (Brisson et al., Nature 310:511-514. (1984)) may be used alone or in combination with the omega leader sequence from TMV (Takamatsu et al., EMBO J., 6:307-311, (1987)). Alternatively, plant promoters such as the small subunit of RUBISCO (Coruzzi et al., EMBO J. 3:1671-1680, (1984); Broglie et al., Science 224:838-843, (1984)); or heat shock promoters (Winter J and Sinibaldi R. M., Results Probl. Cell Differ., 17:85-105, (1991)) may be used. These constructs can be introduced into plant cells by direct DNA transformation or pathogen-mediated transfection. For reviews of such techniques, see Hobbs S. or Murry L. E. (1992) in McGraw Hill Yearbook of Science and Technology, McGraw Hill, New York, N.Y., pp 191-196; or Weissbach and Weissbach (1988) Methods for Plant Molecular Biology, Academic Press, New York, N.Y., pp 421-463.

TRAIL variant product may also be expressed in an insect system. In one such system, Autographa californica nuclear polyhedrosis virus (AcNPV) is used as a vector to express foreign genes in Spodoptera frugiperda cells or in Trichoplusia larvae. The TRAIL variant product coding sequence may be cloned into a nonessential region of the virus, such as the polyhedrin gene, and placed under control of the polyhedrin promoter. Successful insertion of TRAIL variant coding sequence will render the polyhedrin gene inactive and produce recombinant virus lacking coat protein coat. The recombinant viruses are then used to infect S. frugiperda cells or Trichoplusia larvae in which variant protein is expressed (Smith et al., J. Virol. 46:584, (1983); Engelhard, E. K. et al., Proc. Nat. Acad. Sci. 91:3224-7, (1994)).

In mammalian host cells, a number of viral-based expression systems may be utilized. In cases where an adenovirus is used as an expression vector, a TRAIL variant product coding sequence may be ligated into an adenovirus transcription/translation complex consisting of the late promoter and tripartite leader sequence. Insertion in a nonessential E1 or E3 region of the viral genome will result in a viable virus capable of expressing variant protein in infected host cells (Logan and Shenk, Proc. Natl. Acad. Sci. 81:3655-59, (1984). In addition, transcription enhancers, such as the Rous sarcoma virus (RSV) enhancer, may be used to increase expression in mammalian host cells.

Specific initiation signals may also be required for efficient translation of a variant product coding sequence. These signals include the ATG initiation codon and adjacent sequences. In cases where TRAIL variant product coding sequence, its initiation codon and upstream sequences are inserted into the appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf, D. et al, (1994) Results Probl. Cell Differ., 20:125-62, (1994); Bittner et al., Methods in Enzymol 153:516-544, (1987)).

The TRAIL variants of the invention were expressed in CHO cells myeloma cells and E.coli cells produced TRAIL peptides.

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., and Battey, I. (1986) Basic Methods in Molecular Biology). Cell-free translation systems can also be employed to produce polypeptides using RNAs derived from the DNA constructs of the present invention.

A host cell strain may be chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing which cleaves a “pre-pro” form of the protein may also be important for correct insertion, folding and/or function. Different host cells such as CHO, HeLa, MDCK, 293, W138, etc. have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines which stably express variant product may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clumps of stably transformed cells can be proliferated using tissue culture techniques appropriate to the cell type.

Any number of selection systems may be used to recover transformed cell lines. These include, but are not limited to, the herpes simplex virus thymidine kinase (Wigler M., et al., Cell 11:223-32, (1977)) and adenine phosphoribosyltransferase (Lowy I., et al., Cell 22:817-23, (1980)) genes which can be employed in tk- or aprt-cells, respectively. Also, antimetabolite, antibiotic or herbicide resistance can be used as the basis for selection; for example, dhfr which confers resistance to methotrexate (Wigler M., et al., Proc. Natl. Acad. Sci. 77:3567-70, (1980)); npt, which confers resistance to the aminoglycosides neomycin and G-418 (Colbere-Garapin, F. et al., J. Mol. Biol, 150:1-14, (1981)) and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman S. C. and R. C. Mulligan, Proc. Natl. Acad. Sci. 85:8047-51, (1988)). The use of visible markers has gained popularity with such markers as anthocyanins, beta-glucuronidase and its substrate, GUS, and luciferase and its substrates, luciferin and ATP, being widely used not only to identify transformants, but also to quantify the amount of transient or stable protein expression attributable to a specific vector system (Rhodes, C. A. et. al., Methods Mol. Biol., 55:121-131, (1995)).

Host cells transformed with a nucleotide sequence encoding TRAIL variant product may be cultured under conditions suitable for the expression and recovery of the encoded protein from cell culture. The product produced by a recombinant cell may be secreted or contained intracellularly depending on the sequence and/or the vector used. As will be understood by those of skill in the art, expression vectors containing nucleic acid sequences encoding TRAIL variant product can be designed with signal sequences which direct secretion of TRAIL variant product through a prokaryotic or eukaryotic cell membrane.

The TRAIL variant product may also be expressed as a recombinant protein with one or more additional polypeptide domains added to facilitate protein purification. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp, Seattle, Wash.). The inclusion of a protease-cleavable polypeptide linker sequence between the purification domain and TRAIL variant product is useful to facilitate purification. One such expression vector provides for expression of a fusion protein compromising a variant polypeptide fused to a polyhistidine region separated by an enterokinase cleavage site. The histidine residues facilitate purification on IMIAC (immobilized metal ion affinity chromatography, as described in Porath, et al., Protein Expression and Purification, 3:263-281, (1992)) while the enterokinase cleavage site provides a means for isolating variant polypeptide from the fusion protein. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Following transformation of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents, or other methods, which are well know to those skilled in the art.

The TRAIL variant products can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Protein refolding steps can be used, as necessary, in completing configuration of the mature protein. Finally, high performance liquid chromatography (HPLC) can be employed for final purification steps.

C. Diagnostic Applications Utilizing Nucleic Acid Sequences

The nucleic acid sequences of the present invention may be used for a variety of diagnostic purposes. The nucleic acid sequences may be used to detect and quantitate expression of the TRAIL variant in patient's cells, e.g. biopsied tissues, by detecting the presence of mRNA coding for the TRAIL variants products. Alternatively, the assay may be used to detect soluble variant in the serum or blood. This assay typically involves obtaining total mRNA from the tissue or serum and contacting the mRNA with a nucleic acid probe. The probe is a nucleic acid molecule of at least 20 nucleotides, preferably 20-30 nucleotides, capable of specifically hybridizing with a sequence included within the sequence of a nucleic acid molecule encoding the TRAIL variant product under hybridizing conditions, detecting the presence of mRNA hybridized to the probe, and thereby detecting the expression of variant. This assay can be used to distinguish between absence, presence, and excess expression of TRAIL variants product and to monitor levels of variants expression during therapeutic intervention. In addition, the assay may be used to compare the levels of the TRAIL variant of the invention to the levels of the original TRAIL sequence from which it has been varied or to levels of other variants, which comparison may have some physiological meaning.

The invention also contemplates the use of the nucleic acid sequences as a diagnostic for diseases resulting from inherited defective variant sequences, or diseases in which the ratio of the amount of the original TRAIL sequence from which the TRAIL variants was varied to the novel variants of the invention is altered. These sequences can be detected by comparing the sequences of the defective (i.e., mutant) TRAIL variant coding region with that of a normal coding region. Association of the sequence coding for mutant TRAIL variant product with abnormal variant product activity may be verified. In addition, sequences encoding mutant TRAIL variant products can be inserted into a suitable vector for expression in a functional assay system (e.g., colorimetric assay, complementation experiments in a variant protein deficient strain of HEK293 cells) as yet another means to verify or identify mutations. Once mutant genes have been identified, one can then screen populations of interest for carriers of the mutant gene.

Individuals carrying mutations in the nucleic acid sequence of the present invention may be detected at the DNA level by a variety of techniques. Nucleic acids used for diagnosis may be obtained from a patient's cells, including but not limited to such as from blood, urine, saliva, placenta, tissue biopsy and autopsy material. Genomic DNA may be used directly for detection or may be amplified enzymatically by using PCR (Saiki, et al., Nature 324:163-166, (1986)) prior to analysis. RNA or cDNA may also be used for the same purpose. As an example, PCR primers complementary to the nucleic acid of the present invention can be used to identify and analyze mutations in the gene of the present invention. Deletions and insertions can be detected by a change in size of the amplified product in comparison to the normal genotype.

Point mutations can be identified by hybridizing amplified DNA to radiolabeled RNA of the invention or alternatively, radiolabeled antisense DNA sequences of the invention. Sequence changes at specific locations may also be revealed by nuclease protection assays, such RNase and SI protection or the chemical cleavage method (e.g. Cotton, et al Proc. NatL. Acad. Sci. USA, 85:4397-4401, (1985)), or by differences in melting temperatures. “Molecular beacons” (Kostrikis L. G. et al., Science 279:1228-1229, (1998)), hairpin-shaped, single-stranded synthetic oligo-nucleotides containing probe sequences which are complementary to the nucleic acid of the present invention, may also be used to detect point mutations or other sequence changes as well as monitor expression levels of variant product. Such diagnostics would be particularly useful for prenatal testing.

Another method for detecting mutations uses two DNA probes which are designed to hybridize to adjacent regions of a target, with abutting bases, where the region of known or suspected mutation(s) is at or near the abutting bases. The two probes may be joined at the abutting bases, e.g., in the presence of a ligase enzyme, but only if both probes are correctly base paired in the region of probe junction. The presence or absence of mutations is then detectable by the presence or absence of ligated probe.

Also suitable for detecting mutations in the TRAIL variant product coding sequence are oligonucleotide array methods based on sequencing by hybridization (SBH), as described, for example, in U.S. Pat. No. 5,547,839. In a typical method, the DNA target analyte is hybridized with an array of oligonucleotides formed on a microchip. The sequence of the target can then be “read” from the pattern of target binding to the array.

D. Gene Mapping Utilizing Nucleic Acid Sequences

The nucleic acid sequences of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

Briefly, sequences can be mapped to chromosomes by preparing PCR primers (preferably 20-30 bp) from the variant 5′ cDNA. Computer analysis of the 3′ untranslated region is used to rapidly select primers that do not span more than one exon in the genomic DNA, which would complicate the amplification process. These primers are then used for PCR screening of somatic cell hybrids containing individual human chromosomes. Only those hybrids containing the human gene corresponding to the primer will yield an amplified fragment.

PCR mapping of somatic cell hybrids or using instead radiation hybrids are rapid procedures for assigning a particular DNA to a particular chromosome. Using the present invention with the same oligonucleotide primers, sublocalization can be achieved with panels of fragments from specific chromosomes or pools of large genomic clones in an analogous manner. Other mapping strategies that can similarly be used to map to its chromosome include in situ hybridization, prescreening with labeled flow-sorted chromosomes and preselection by hybridization to construct chromosome specific-cDNA libraries.

Fluorescence in situ hybridization (FISH) of a cDNA clone to a metaphase chromosomal spread can be used to provide a precise chromosomal location in one step. This technique can be used with cDNA as short as 50 or 60 bases. For a review of this technique, see Verma et al., Human Chromosomes: a Manual of Basic Techniques, (1988) Pergamon Press, New York.

Once a sequence has been mapped to a precise chromosomal location, the physical position of the sequence on the chromosome can be correlated with genetic map data. Such data are found, for example, in the OMIM database (Center for Medical Genetics, Johns Hopkins University, Baltimore, MD and National Center for Biotechnology Information, National Library of Medicine, Bethesda, Md.). The OMIM gene map presents the cytogenetic map location of disease genes and other expressed genes. The OMIM database provides information on diseases associated with the chromosomal location. Such associations include the results of linkage analysis mapped to this interval, and the correlation of translocations and other chromosomal aberrations in this area with the advent of polygenic diseases, such as cancer, in general and prostate cancer in particular.

E. Therapeutic Applications of Nucleic Acid Sequences

Nucleic acid sequences of the invention may also be used for therapeutic purposes. Turning first to the second aspect of the invention (i.e. inhibition of expression of TRAIL variant), expression of TRAIL variant product may be modulated through antisense technology, which controls gene expression through hybridization of complementary nucleic acid sequences, i.e. antisense DNA or RNA, to the control, 5′ or regulatory regions of the gene encoding variant product. For example, the 5′ coding portion of the nucleic acid sequence sequence which codes for the product of the present invention is used to design an antisense oligonucleotide of from about 10 to 40 base pairs in length. Oligonucleotides derived from the transcription start site, e.g. between positions −10 and +10 from the start site, are preferred. An antisense DNA oligonucleotide is designed to be complementary to a region of the nucleic acid sequence involved in transcription (Lee et al., Nucl. Acids, Res., 6:3073, (1979); Cooney et al., Science 241:456, (1988); and Dervan et al., Science 251:1360, (1991)), thereby preventing transcription and the production of the variant products. An antisense RNA oligonucleotide hybridizes to the mRNA in vivo and blocks translation of the mRNA molecule into the variant products (Okano J. Neurochem. 56:560, (1991)). The antisense constructs can be delivered to cells by procedures known in the art such that the antisense RNA or DNA may be expressed in vivo. The antisense may be antisense mRNA or DNA sequence capable of coding such antisense mRNA. The antisense mRNA or the DNA coding thereof can be complementary to the full sequence of nucleic acid sequences coding for the TRAIL variant protein or to a fragment of such a sequence which is sufficient to inhibit production of a protein product.

Turning now to the first aspect of the invention, i.e. expression of TRAIL variant, expression of TRAIL variant product may be increased by providing coding sequences for coding for said product under the control of suitable control elements ending its expression in the desired host.

The nucleic acid sequences of the invention may be employed in combination with a suitable pharmaceutical carrier. Such compositions comprise a therapeutically effective amount of the compound, and a pharmaceutically acceptable carrier or excipient. Such a carrier includes but is not limited to saline, buffered saline, dextrose, water, glycerol, ethanol, and combinations thereof. The formulation should suit the mode of administration.

The products of the invention as well as any activators and deactivators compounds (see below) which are polypeptides, may also be employed in accordance with the present invention by expression of such polypeptides in vivo, which is often referred to as “gene therapy.” Cells from a patient may be engineered with a nucleic acid sequence (DNA or RNA) encoding a polypeptide ex vivo, with the engineered cells then being provided to a patient to be treated with the polypeptide. Such methods are well-known in the art. For example, cells may be engineered by procedures known in the art by use of a retroviral particle containing RNA encoding a polypeptide of the present invention.

Similarly, cells may be engineered in vivo for expression of a polypeptide in vivo by procedures known in the art. As known in the art, a producer cell for producing a retroviral particle containing RNA encoding the polypeptide of the present invention may be administered to a patient for engineering cells in vivo and expression of the polypeptide in vivo. These and other methods for administering a product of the present invention by such method should be apparent to those skilled in the art from the teachings of the present invention. For example, the expression vehicle for engineering cells may be other than a retrovirus, for example, an adenovirus which may be used to engineer cells in vivo after combination with a suitable delivery vehicle.

Retroviruses from which the retroviral plasmid vectors mentioned above may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PE501, PA317, psi-2, psi-AM, PA12, T19-14X, VT-19-17-H2, psi-CRE, psi-CRIP, GP+E-86, GP+envAm12, and DAN cell lines as described in Miller (Human Gene Therapy, Vol. 1, pg. 5-14, (1990)). The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

The genes introduced into cells may be placed under the control of inducible promoters, such as the radiation-inducible Egr-1 promoter, (Maceri, H. J., et al., Cancer Res., 56(19):4311 (1996)), to stimulate variant production or antisense inhibition in response to radiation, eg., radiation therapy for treating tumors.

### EXAMPLE II

### TRAIL Variant Product

The substantially purified TRAIL variant product of the invention has been defined above as the product coded from the nucleic acid sequence of the invention. Preferably the amino acid sequence is an amino acid sequence having at least 90% identity to any one of the sequences identified as SEQ ID NO:3 or SEQ ID NO:4 provided that the amino acid sequence is not identical to that of the original sequence from which it has been varied. The protein or polypeptide may be in mature and/or modified form, also as defined above. Also contemplated are protein fragments having at least 10 contiguous amino acid residues, preferably at least 10-20 residues, derived from the TRAIL variant product, as well as homologues as explained above.

The sequence variations are preferably those that are considered conserved substitutions, as defined above. Thus, for example, a protein with a sequence having at least 90% sequence identity with any of the products identified as SEQ ID NO:3 or 4, preferably by utilizing conserved substitutions as defined above is also part of the invention, and provided that it is not identical to the original peptide from which it has been varied. The TRAIL variant product may be (i) one in which one or more of the amino acid residues in a sequence listed above are substituted with a conserved or non-conserved amino acid residue (preferably a conserved amino acid residue), or (ii) one in which one or more of the amino acid residues includes a substituent group, or (iii) one in which the TRAIL variant product is fused with another compound, such as a compound to increase the half-life of the protein (for example, polyethylene glycol (PEG)), or a moiety which serves as targeting means to direct the protein to its target tissue or target cell population (such as an antibody), or (iv) one in which additional amino acids are fused to the TRAIL variant product. Such fragments, variants and derivatives are deemed to be within the scope of those skilled in the art from the teachings herein.

As indicated above in connection with the TRAIL variants SEQ ID NOS:1, 2, 3, 4 and 7 lack the conserved TNF domain while SEQ ID NOS:5, 6 and 7 lack part but not all of the TNF conserved domain while keeping the reading frame.

A. Preparation of TRAIL Variants Products

Recombinant methods for producing and isolating the TRAIL variant product, and fragments of the protein are described above.

In addition to recombinant production, fragments and portions of variant product may be produced by direct peptide synthesis using solid-phase techniques (cf. Stewart et al., (1969) Solid-Phase Peptide Synthesis, WH Freeman Co, San Francisco; Merrifield J., J. Am. Chem. Soc., 85:2149-2154, (1963)). In vitro peptide synthesis may be performed using manual techniques or by automation. Automated synthesis may be achieved, for example, using Applied Biosystems 431A Peptide Synthesizer (Perkin Elmer, Foster City, Calif.) in accordance with the instructions provided by the manufacturer. Fragments of TRAIL variant product may be chemically synthesized separately and combined using chemical methods to produce the full length molecule.

B. Therapeutic Uses and Compositions Utilizing the TRAIL Variants Products

The TRAIL variants products of the invention is generally useful in treating diseases and disorders which are characterized by a lower than normal level of TRAIL variant expression, and or diseases which can be cured or ameliorated by raising the level of the TRAIL variant product, even if the level is normal. The variant aspect are diseases where a beneficial effect may be achieved by producing a cytotoxic effect (for example, due to apoptosis) in a target cell population, notably in cancer cells).

TRAIL variant products or fragments may be administered by any of a number of routes and methods designed to provide a consistent and predictable concentration of compound at the target organ or tissue. The product-containing compositions may be administered alone or in combination with other agents, such as stabilizing compounds, and/or in combination with other pharmaceutical agents such as drugs or hormones.

TRAIL variant product-containing compositions may be administered by a number of routes including, but not limited to oral, intravenous, intramuscular, transdermal, subcutaneous, topical, sublingual, or rectal means as well as by nasal application. TRAIL variant product-containing compositions may also be administered via liposomes. Such administration routes and appropriate formulations are generally known to those of skill in the art.

The TRAIL variant product can be given via intravenous or intraperitoneal injection. Similarly, the product may be injected to other localized regions of the body. The product may also be administered via nasal insufflation. Enteral administration is also possible. For such administration, the product should be formulated into an appropriate capsule or elixir for oral administration, or into a suppository for rectal administration.

The foregoing exemplary administration modes will likely require that the product be formulated into an appropriate carrier, including ointments, gels, suppositories. Appropriate formulations are well known to persons skilled in the art.

Dosage of the product will vary, depending upon the potency and therapeutic index of the particular polypeptide selected.

A therapeutic composition for use in the treatment method can include the product in a sterile injectable solution, the polypeptide in an oral delivery vehicle, the product in an aerosol suitable for nasal administration, or the product in a nebulized form, all prepared according to well known methods. Such compositions comprise a therapeutically effective amount of the compound, and a pharmaceutically acceptable carrier or excipient. Such a carrier includes but is not limited to saline, buffered saline, dextrose, water, glycerol, ethanol, and combinations thereof. The product of the invention may also be used to modulate endothelial differentiation and proliferation as well as to modulate apoptosis either ex vivo or in vitro, for example, in cell cultures.

### EXAMPLE III

### Screening Methods for Agonists and Antagonists (Inhibitors)

The present invention also includes an assay for identifying molecules, such as synthetic drugs, antibodies, peptides, or other molecules, which have a modulating effect on the activity of the TRAIL variant product, e.g. agonists or antagonists (or inhibitors) of the TRAIL variant products of the present invention. Such an assay comprises the steps of providing a TRAIL variants products encoded by the nucleic acid sequences of the present invention, contacting the TRAIL variant protein with one or more candidate molecules to determine the candidate molecules modulating effect on the activity of the variant product, and selecting from the molecules a candidate's molecule capable of modulating TRAIL variant product physiological activity.

The TRAIL variant product, its catalytic or immunogenic fragments or oligopeptides thereof, can be used for screening therapeutic compounds in any of a variety of drug screening techniques. The fragment employed in such a test may be free in solution, affixed to a solid support, borne on a cell membrane or located intracellularly. The formation of binding complexes, between variant product and the agent being tested, may be measured. Alternatively, the agonist or antagonist (inhibitor) may work by serving as agonist or antagonist, respectively, of the TRAIL variant, or by binding the native ligand of the TRAIL receptor, and their effect may be determined in connection with any of the above.

Another technique for drug screening which may be used provides for high throughput screening of compounds having suitable binding affinity to the TRAIL variant product is described in detail by Geysen in PCT Application WO 84/03564, published on Sep. 13, 1984. In summary, large numbers of different small peptide test compounds are synthesized on a solid substrate, such as plastic pins or some other surface. The peptide test compounds are reacted with the full TRAIL variant product or with fragments of TRAIL variant product and washed. Bound TRAIL variant product is then detected by methods well known in the art. Substantially purified TRAIL variant product can also be coated directly onto plates for use in the aforementioned drug screening techniques. Alternatively, non-neutralizing antibodies can be used to capture the peptide and immobilize it on a solid support.

Antibodies to the variant product, as described in Example V below, may also be used in screening assays according to methods well known in the art. For example, a “sandwich” assay may be performed, in which an anti-variant antibody is affixed to a solid surface such as a microtiter plate and variant product is added. Such an assay can be used to capture compounds which bind to the variant product. Alternatively, such an assay may be used to measure the ability of compounds to influence with the binding of the TRAIL variant product to the variant receptor, and then select those compounds which effect the binding.

### EXAMPLE IV

### Anti-variant Antibodies/Distinguishing Antibodies

A. Synthesis

In still another aspect of the invention, the purified variant product is used to produce anti-variant antibodies which have diagnostic and therapeutic uses related to the activity, distribution, and expression of the TRAIL variants products. As indicated above, the antibodies may also be directed solely to sequences present only in the original TRAIL sequence but not in the TRAIL variant (“distinguishing antibodies”).

Antibodies to the TRAIL variant product or to the distinguishing sequence present only in the original TRAIL and not in the TRAIL variant (the latter termed “distinguishing antibodies”) may be generated by methods well known in the art. Such antibodies may include, but are not limited to, polyclonal, monoclonal, chimeric, humanized, single chain, Fab fragments and fragments produced by an Fab expression library. Antibodies, i.e., those which inhibit dimer formation, are especially preferred for therapeutic use.

A fragment of the TRAIL variant product for antibody induction is not required to feature biological activity but has to feature immunological activity; however, the protein fragment or oligopeptide must be antigenic. Peptides used to induce specific antibodies may have an amino acid sequence consisting of at least five amino acids, preferably at least 10 amino acids of the sequences specified in any one of SEQ ID NO:9 or SEQ ID NO:16 or in distinguishing sequences present only in the TRAIL variant or only in the original TRAIL sequence as explained above. Preferably they should mimic a portion of the amino acid sequence of the natural protein and may contain the entire amino acid sequence of a small, naturally occurring molecule. Short stretches of TRAIL variant protein amino acids may be fused with those of another protein such as keyhole limpet hemocyanin and antibody produced against the chimeric molecule. Procedures well known in the art can be used for the production of antibodies to TRAIL variant product.

For the production of antibodies, various hosts including goats, rabbits, rats, mice, etc may be immunized by injection with TRAIL variant product or any portion, fragment or oligopeptide which retains immunogenic properties. Depending on the host species, various adjuvants may be used to increase immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Monoclonal antibodies to TRAIL variant protein may be prepared using any technique which provides for the production of antibody molecules by continuous cell lines in culture. These include but are not limited to the hybridoma technique originally described by Koehler and Milstein (Nature 256:495-497, (1975)), the human B-cell hybridoma technique (Kosbor et al., Immunol. Today 4:72, (1983); Cote et al., Proc. Natl. Acad. Sci. 80:2026-2030, (1983)) and the EBV-hybridoma technique (Cole, et al., Mol. Cell Biol. 62:109-120, (1984)).

Techniques developed for the production of “chimeric antibodies”, the splicing of mouse antibody genes to human antibody genes to obtain a molecule with appropriate antigen specificity and biological activity can also be used (Morrison et al., Proc. Natl. Acad. Sc. 81:6851-6855, (1984); Neuberger et al., Nature 312:604-608, (1984); Takeda et al., Nature 314:452-454, (1985)). Alternatively, techniques described for the production of single chain antibodies (U.S. Pat. No. 4,946,778) can be adapted to produce single-chain antibodies specific for the variant protein.

Antibodies may also be produced by inducing in vivo production in the lymphocyte population or by screening recombinant immunoglobulin libraries or panels of highly specific binding reagents as disclosed in Orlandi et al. (Proc. Natl. Acad. Sci. 86:3833-3837, 1989)), and Winter G and Milstein C., (Nature 349:293-299, (1991)).

Antibody fragments which contain specific binding sites for the TRAIL variant protein may also be generated. For example, such fragments include, but are not limited to, the F(ab′)₂ fragments which can be produced by pepsin digestion of the antibody molecule and the Fab fragments which can be generated 5 by reducing the disulfide bridges of the F(ab′)₂ fragments. Alternatively, Fab expression libraries may be constructed to allow rapid and easy identification of monoclonal Fab fragments with the desired specificity (Huse W. D. et al., Science 256:1275-1281, (1989)).

B. Diagnostic Applications of Antibodies

A variety of protocols for competitive binding or immunoradiometric assays using either polyclonal or monoclonal antibodies with established specificities are well known in the art. Such immunoassays typically involve the formation of complexes between the TRAIL variant product and its specific antibody and the measurement of complex formation. A two-site, monoclonal-based immunoassay utilizing monoclonal antibodies reactive to two noninterfering epitopes on a specific variant product is preferred, but a competitive binding assay may also be employed. These assays are described in Maddox D. E., et al., (J. Exp. Med. 158:1211, (1983)).

Antibodies which specifically bind the TRAIL variant product or distinguishing antibodies which bind to sequences which distinguish the TRAIL variant from the original TRAIL sequence (as explained above) are useful for the diagnosis of conditions or diseases characterized by expression of the novel TRAIL variant of the invention (where normally it is not expressed) by over or under expression of TRAIL variants as well as for detection of diseases in which the proportion between the amount of the TRAIL variants of the invention and the original TRAIL sequence from which it varied is altered. Alternatively, such antibodies may be used in assays to monitor patients being treated with TRAIL variants products, its agonists, or its antagonists. Diagnostic assays for variant protein include methods utilizing the antibody and a label to detect variant product in human body fluids or extracts of cells or tissues. The products and antibodies of the present invention may be used with or without modification. Frequently, the proteins and antibodies will be labeled by joining them, either covalently or noncovalently, with a reporter molecule. A wide variety of reporter molecules are known in the art.

A variety of protocols for measuring the TRAIL variant product, using either polyclonal or monoclonal antibodies specific for the respective protein are known in the art. Examples include enzyme-linked immunosorbent assay (ELISA), radioimmunoassay (RIA), and fluorescent activated cell sorting (FACS). As noted above, a two-site, monoclonal-based immunoassay utilizing monoclonal antibodies reactive to two non-interfering epitopes on TRAIL variant product is preferred, but a competitive binding assay may be employed. These assays are described, among other places, in Maddox, et a. (supra). Such protocols provide a basis for diagnosing altered or abnormal levels of TRAIL variant product expression. Normal or standard values for TRAIL variant product expression are established by combining body fluids or cell extracts taken from normal subjects, preferably human, with antibodies to TRAIL variants products under conditions suitable for complex formation which are well known in the art. The amount of standard complex formation may be quantified by various methods, preferably by photometric methods. Then, standard values obtained from normal samples may be compared with values obtained from samples from subjects potentially affected by disease. Deviation between standard and subject values establishes the presence of disease state.

The antibody assays are useful to determine the level of TRAIL variants products present in a body fluid sample, in order to determine whether it is being expressed at all, whether it is being overexpressed or underexpressed in the tissue, or as an indication of how TRAIL variants levels of variable products are responding to drug treatment.

C. Therapeutic Uses of Antibodies

In addition to their diagnostic use the antibodies may have a therapeutical utility in blocking or decreasing the activity of the TRAIL variant product in pathological conditions where beneficial effect can be achieved by such a decrease. Again, distinguishing antibodies may be used to neutralize differentially either the TRAIL variant or the original sequence as the case may be.

The antibody employed is preferably a humanized monoclonal antibody, or a human Mab produced by known globulin-gene library methods. The antibody is administered typically as a sterile solution by IV injection, although other parenteral routes may be suitable. Typically, the antibody is administered in an amount between about 1-15 mg/kg body weight of the subject. Treatment is continued, e.g., with dosing every 1-7 days, until a therapeutic improvement is seen.

Although the invention has been described with reference to specific methods and embodiments, it is appreciated that various modifications and changes may be made without departing from the invention.

1. An isolated nucleic acid sequence, of an alternative splicing variant of TNF-related apoptosis inducing ligand (TRAIL), selected from the group consisting of:
(i) the nucleic acid sequence depicted in any one of SEQ ID NO: 1 to SEQ ID NO: 8;
(ii) nucleic acid sequences having at least 90% identity with the sequence of (i); and
(iii) fragments of (i) or (ii) of at least 20 b.p., provided that said fragment contains a sequence which is not present, as a continuous stretch of nucleotides, in the original nucleic acid sequence of TRAIL from which the sequences of (i) have been varied by alternative splicing.

2. An isolated nucleic acid sequence complementary to the nucleic acid sequence of claim 1.

3. An amino acid sequence selected from the group consisting of:
(i) an amino acid sequence coded by the isolated nucleic acid sequence of alternative splice variants of claim 1;
(ii) homologues of the amino acid sequences of (i) in which one or more amino acids has been added, deleted, replaced or chemically modified in the region, or adjacent to the region, where the amino acid sequences differs from the original amino acid sequence, coded by the original TRAIL nucleic acid sequence from which the variant has been varied by alternative splicing.

4. An amino acid sequence according to claim 3, as depicted in any one of SEQ ID NO:9 to SEQ ID NO:16.

5. An isolated nucleic acid sequence coding for any one of the amino acid sequences of claim 3 or 4.

6. A purified antibody which binds specifically to any of the amino acid sequence of claim 3 or 4.

7. A purified antibody which binds to an amino acid sequence which is present in the original TRAIL sequence but is not present in the amino acid sequence of claims 3 or 4.

8. An expression vector comprising any one of the nucleic acid sequences of claim 1 or 5 and control elements for the expression of the nucleic acid sequence in a suitable host.

9. An expression vector comprising any one of the nucleic acid sequences of claim 2, and control elements for the expression of the nucleic acid sequences in a suitable host.

10. A host cell transfected by the expression vector of claim 8 or 9.

11. A pharmaceutical composition comprising a pharmaceutically acceptable carrier and as an active ingredient an agent selected from the group consisting of:
(i) the expression vector of claim 8; and
(ii) any one of the amino acid sequences of claim 3 or 4.

12. A pharmaceutical composition according to claim 11, for treatment of diseases which can be ameliorated, cured or prevented by raising the level of any one of the amino acid sequences depicted in SEQ ID NO:9 to SEQ ID NO:16.

13. A pharmaceutical composition according to claim 11, for causing a cytotoxic effect in a target cell population.

14. A pharmaceutical composition according to claim 13, wherein the cell population is cancer cells.

15. A pharmaceutical composition comprising a pharmaceutically acceptable carrier and as an active ingredient an agent selected from the group consisting of:
(i) any one of the nucleic acid sequences of claim 2;
(ii) the expression vector of claim 9; and
(iii) the purified antibody of claim 6.

16. A pharmaceutical composition according to claim 15, for treatment of diseases which can be ameliorated, cured or prevented by lowering the level of any one of the amino acid sequences depicted in SEQ ID NO:9 to SEQ ID NO:16.

17. A pharmaceutical composition according to claim 16, wherein the disease is manifested by undesired death of cells.

18. A method for detecting the presence of a variant nucleic acid sequence of TRAIL variant in a biological sample, comprising the steps of:
(a) hybridizing to nucleic acid material of said biological sample any one of the nucleic acid sequences of claim 1 or 2; and
(b) detecting hybridization complex;
wherein the presence of said hybridization complex correlates with the presence of an variant nucleic acid sequence in the said biological sample.

19. A method for determining the level of variant nucleic acid sequences of TRAIL in a biological sample comprising the steps of:
(a) hybridizing to nucleic acid material of said biological sample any one of the nucleic acid sequences of claim 1 or 2; and
(b) determining the amount of hybridization complexes and normalizing said amount to provide the level of the variant nucleic acid sequences in the sample.

20. A method for determining the ratio between the level of the nucleic acid sequence of a TRAIL variant in a first biological sample and the level of the original TRAIL sequence from which the variant has been varied by alternative splicing, in a second biological sample comprising:
(a) determining the level of the TRAIL variant nucleic acid sequence in the first biological sample according to the method of claim 19;
(b) determining the level of the TRAIL original sequence in the second biological sample; and
(c) comprising the levels obtained in (a) and (b) to give said ratio.

21. A method according to claim 20, wherein said first and said second biological samples are the same sample.

22. A method according to any of claims 18 to 21, wherein the nucleic acid material of said biological sample are mRNA transcripts.

23. A method according to claim 22, where the nucleic acid sequence is present in a nucleic acid chip.

24. A method for identifying candidate compounds capable of binding to the variant product and modulating its activity the method comprising:
(i) providing any one of the amino acid sequences as defined in claim 3 or 4;
(ii) contacting a candidate compound with said amino acid sequence;
(iii) determining the effect of said candidate compound on the biological activity of said protein or polypeptide and selecting those compounds which show a significant effect on said biological activity.

25. A method according to claim 24, wherein the compound is an agonist and the measured effect is increase in the biological activity.

26. A method according to claim 24, wherein the compound is an antagonist and the effect is decrease in the biological activity.

27. An agonist of any one of the amino acid sequences of claim 3 or 4.

28. An antagonist of any one of the amino acid sequences of claims 3 or 4.

29. A method for detecting any one of the amino acid sequences of claim 3 or 4 in a biological sample, comprising the steps of:.
(a) contacting with said biological sample the antibody of claim 6, thereby forming an antibody-antigen complex; and
(b) detecting said antibody-antigen complex
wherein the presence of said antibody-antigen complex correlates with the presence of the desired amino acid in said biological sample.

30. A method for detecting the level of the amino acid sequence of any one of claim 3 or 4 in a biological sample, comprising the steps of:
(a) contacting with said biological sample the antibody of claim 6, thereby forming an antibody-antigen complex; and
(b) detecting the amount of said antibody-antigen complex and normalizing said amount to provide the level of said amino acid sequence in the sample.

31. A method for determining the ratio between the level of any one of the amino acid sequences of claims 3 or 4 of variant TRAIL present in a first biological sample and the level of the original TRAIL amino acid, present in a second biological sample, the method comprising:
(a) determining the level of the amino acid sequences of claims 3 or 4 into a first sample by the method of claim 30;
(b) determining the level of the original TRAIL amino acid sequence in the second sample; and
(d) comparing the level obtained in (a) and (b) to give said ratio.

32. A method according to claim 31, wherein said first and said second biological samples are the same sample.
